(19) United States
(12) Patent Application Publication
(10) Pub. No.: US 2025/0198919 A1
(43) Pub. Date: Jun. 19, 2025

(54) DEVICE AND METHOD FOR MEASURING HYDROXYL CONTENT IN HYDROCARBON FUEL

(71) Applicant: EMCEE ELECTRONICS, INC., Venice, FL (US)

(72) Inventor: Ryan Matthew Case, Sarasota, FL (US)

(21) Appl. No.: 18/982,260

(22) Filed: Dec. 16, 2024

Related U.S. Application Data
(60) Provisional application No. 63/610,770, filed on Dec. 15, 2023.

(51) Int. Cl. G01N 21/359; G01N 27/02; G01N 33/22

(52) U.S. Cl. CPC G01N 21/359; G01N 27/026; G01N 33/22; G01N 2201/0221; G01N 2201/1296

(57) ABSTRACT

A device for measuring hydroxyl-containing compounds in hydrocarbon samples combines near-infrared (NIR) spectroscopy and electrochemical impedance (EI) spectroscopy to accurately identify and quantify compounds like ethanol, methanol, and water. The portable device includes a sample inlet, NIR spectrometer for compound identification, EI spectrometer for compound differentiation, and a controller that processes signals to determine presence and amounts of hydroxyl-containing compounds. The device incorporates temperature control, a custom syringe pump system, and a quartz-tungsten-halogen light source with analog feedback control. A rechargeable lithium-ion battery provides extended field operation. The controller uses machine learning algorithms to perform real-time chemometrics, achieving measurement accuracy within 0.1% by mass. The briefcase-style configuration includes polyetheretherketone fluid handling components, touch screen interface, wireless connectivity, and environmental protection rated to IP56 standards. This portable solution enables rapid on-site fuel analysis without requiring complex laboratory equipment or specialized operator training.

### CROSS-REFERENCE TO RELATED APPLICATIONS

This application claims the benefit of U.S. Provisional Application No. 63/610,770, filed on Dec. 15, 2023. The entire disclosure of the above application is incorporated herein by reference.

### FIELD

The present technology relates to ways of measuring hydroxyl content in a hydrocarbon fuel, including ways to ascertain the presence and amounts of certain compounds having hydroxyl groups in hydrocarbon fuels, such as gasoline.

### INTRODUCTION

This section provides background information related to the present disclosure which is not necessarily prior art.

In the U.S., federal and state mandates regulate the ethanol concentration in gasoline. Most states mandate an ethanol content in gasoline of 10%. This proportion has recently been approved by the EPA to increase to 15%. There are approximately 145,000 gas stations in the fifty U.S. states alone. There are also numerous independent and government fuel labs, pipelines, storage terminals, and refineries throughout the U.S. and the world. Biofuel (e.g., ethanol) mandates further exist in Europe and many other countries. One result of these mandates is an entire industry devoted to the testing of gasoline. Based on input from only the state of Florida, it is estimated that over a million gasoline tests are performed annually in this one state. Gasoline analysis is therefore necessary in each of the fifty U.S. states, many countries, third-party fuel testing labs, pipelines, storage terminals, and refineries. A significant portion of the energy industry accordingly has an interest in analyzing fuels for composition confirmation, performance, and quality assurance demands.

The only existing method in ASTM's specification for gasoline used to measure the concentration of ethanol is by gas chromatography-mass spectrometry (GC-MS) in a laboratory setting. GC-MS is an expensive and time-consuming process requiring a skilled operator and complex equipment. A technician takes a one-liter sample from a gas pump and sends it to a nearby GC-MS lab for overnight analysis. By the time the results are known, the tested gasoline lot may be long gone. Although GC-MS testing is highly accurate and offers high resolution, it does not provide an indication of the water saturation of the fuel at the pump. This water measurement can be extremely important to fuel performance, because if phase separation occurs, the octane and volatility ratings of the fuel drop, resulting in out-of-specification gasoline. Also, methanol in gasoline is considered a contaminant and should not be present in any amount.

Accordingly, there is a need to quickly, efficiently, and accurately measure the presence and amounts of ethanol, water, and methanol in a gasoline sample. It would be ideal if measurement of these compounds could be performed on-site or in the field, as opposed to sending the sample to a remote testing lab. Reduced costs associated with testing could further facilitate compliance with fuel mandates and ensure fuel performance.

### SUMMARY

In concordance with the instant disclosure, ways of measuring hydroxyl-containing compounds in hydrocarbon samples are provided, including articles of manufacture, systems, and processes that relate to a gasoline analyzer.

Devices and uses thereof in analyzing hydrocarbon fuels, such as gasoline, are provided, which are portable and capable of detecting the presence and amounts of water, ethanol, and methanol in a sample. The present technology uses a combination of near infrared (NIR) spectroscopy and electro-impedance spectroscopy (EI) to accurately identify the concentration of components of a gasoline sample to within about 0.1% by mass. While certain portable lab instruments can measure ethanol content in gasoline, none exhibit the resolution and accuracy of the present technology. Also, the present technology can measure the concentrations of water and methanol in addition to ethanol, unlike other instruments. Certain instruments use absorbance spectroscopy only, and only measure ethanol concentration. Furthermore, certain instruments do not incorporate temperature compensation in their software algorithms, whereas the present technology can be configured to compensate for temperature fluctuations.

Water content of gasoline in storage can change over time due to moisture in the air and the containing vessel. If the water content reaches the water saturation point of the gasoline, ethanol present in the gasoline can come out of solution along with the excess water, rendering the gasoline out of specification. The water saturation point depends on the ethanol concentration and sample temperature.

In certain embodiments, a device for measuring hydroxyl-containing compounds in a hydrocarbon sample can include the following aspects. A sample inlet can receive the hydrocarbon sample. A near-infrared (NIR) spectrometer can provide a first signal relating to identification of hydroxyl-containing compounds in the hydrocarbon sample. An electrochemical impedance (EI) spectrometer can provide a second signal relating to differentiation of hydroxyl-containing compounds in the hydrocarbon sample. A controller can receive the first signal from the NIR spectrometer and the second signal from the EI spectrometer, where the controller can determine a presence and an amount of hydroxyl-containing compounds in the hydrocarbon sample. Embodiments of the device can include a temperature control means configured to control a temperature of the hydrocarbon sample, a syringe configured to be fluidly coupled to the sample inlet, a power source, a light source, an actuator mechanically coupled to the sample inlet and configured to transfer the hydrocarbon sample from the sample inlet to the NIR spectrometer and the EI spectrometer, an electronic user interface, a data port configured to provide input/output signals with the controller, and/or a housing.

In certain embodiments, the device can be used in a method for measuring hydroxyl-containing compounds in a hydrocarbon sample. The hydrocarbon sample can be received in the sample inlet. The first signal can be collected using the NIR spectrometer. The second signal can be collected using the EI spectrometer. The controller can receive the first signal and the second signal, where the controller can then determine the presence and the amount of hydroxyl-containing compounds in the hydrocarbon sample.

The subject device allows for field measurements to be conducted in a matter of minutes by existing field technicians with little additional training. Further benefits include lower costs per measurement and the ability to more closely monitor and react to ethanol/gasoline/water issues. Additional benefits of the subject device include the ability to measure the concentration of environmental contaminants in water or other liquids with a modest modification of components and software.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top perspective view of an embodiment of a device for analyzing a hydrocarbon fuel for the presence of compounds containing hydroxyl groups;

FIG. 2 is a left side elevational view of the device of FIG. 1;

FIG. 3 is a right side elevational view of the device of FIG. 1;

FIG. 4 is top perspective view of the device of FIG. 1, depicted with a lid removed;

FIG. 5 is a top perspective view of the device of FIG. 4, depicted with an access panel hingedly open;

FIG. 6 is a top perspective view of the device of FIG. 5, depicted with an interior panel removed;

FIG. 7 graphically depicts example average absorbance (AU) values for a sample taken over an infrared light wavelength range of 1100-1300 nm; and

FIGS. 8A and 8B graphically depict examples of Bode and Nyquist plots for gasoline samples having ethanol values ranging from 10%-20%.

FIG. 9 is a flowchart depicting an embodiment of a method of measuring hydroxyl-containing compounds in a hydrocarbon sample.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology relates to fuel analysis, including the identification of hydroxyl containing compounds in a hydrocarbon fuel, where devices and methods are provided that allow in-field detection of the presence and amounts of certain compounds having hydroxyl groups, and where the analyzed sample can include a gasoline sample. A device for measuring hydroxyl-containing compounds in a hydrocarbon sample can include the following components: a sample inlet for receiving the hydrocarbon sample; a near-infrared (NIR) spectrometer configured to provide a first signal relating to identification of hydroxyl-containing compounds in the hydrocarbon sample; an electrochemical impedance (EI) spectrometer configured to provide a second signal relating to differentiation of hydroxyl-containing compounds in the hydrocarbon sample; and a controller configured to receive the first signal from the NIR spectrometer and the second signal from the EI spectrometer, the controller configured to determine a presence and an amount of hydroxyl-containing compounds in the hydrocarbon sample.

The subject device and uses thereof allow measurement of the concentrations of hydroxyl-containing compounds (e.g., ethanol, water, and methanol) in a hydrocarbon fuel (e.g., gasoline), at an accuracy and precision comparable to the use of Gas Chromatography-Mass Spectroscopy (GC-MS), where the device is portable enough for field use. The premise behind how the device performs measurements lies behind two characterization techniques—near infrared spectroscopy (NIRS) and electrochemical impedance spectroscopy (EIS). The use of NIRS provides for a means to identify functional groups in the sample for compounds present in concentrations of at least 0.1% by volume; e.g., compounds having hydroxyl (—OH) groups as part of their molecular architecture. The use of EIS provides for a contrast between hydroxyl-containing compounds such as ethanol, methanol, water, and gasoline; e.g., EIS signal response is particularly sensitive to the relative permittivity of the substances in the fuel sample (e.g., 80.1 for water, 33.0 for methanol, 25.3 for ethanol, and about 2.0 for gasoline dielectric constant (DC)). The device can take the results from these two characterization techniques and can use a chemometric model to determine a reading for a particular sample.

In order for the subject device to have the capacity to measure concentrations with accuracy and precision comparable to the use of GC-MS, the present device is configured with the following certain aspects. The absorbance (which comes from the NIRS data), permittivity, permeability, and electrical conductivity of the fuel sample (which are enveloped in the EIS data) depend on temperature, so a means of controlling the temperature of the sample as it is being tested is provided, as the temperature of the sample can vary depending on local meteorological conditions. The fuel sample can pass through a collection of flow-throughs at a uniform rate, and in order to accommodate the testing regions of the instrument, a custom flow cell is provided. The light that is to go through the fuel sample can be collimated, and the device's signal to background ratio is sensitive to how much of that incident light interacts with the sample, so a custom optical setup is provided. The EIS chip can be highly susceptible to cross contamination from sample to sample, so the provided EIS chip can be disposable, and in order to keep the cost per test to a minimum, a specially configured EIS chip is provided herein for the subject device. Software used to run the device can accept upgrades following different or more refined models for interpreting sensor data to measurement values are developed, and as the device is used for testing other types of samples.

The present technology can provide accuracy and precision comparable to GC-MS measurement of a fuel sample. The subject device can be used in environments having wide temperature deviations and can take into account temperature-based changes in accuracy and precision, in embodiments where the behavior of one or more of the device sensors is temperature dependent. The device can incorporate sufficient thermal insulation to the electronic components, sensors, and the fuel sample lines to mitigate deviation in measurements, as necessary. The subject device can also efficiently process a hydrocarbon fuel sample and be purged of the hydrocarbon fuel sample to minimize any residue or vapors within the device. This can allow subsequent sampling and analysis without contamination from previous samples, as well as minimize any aspects related to undesired hydrocarbon residues and/or vapors. To this end, the device can include sealed pumps, lines, and vapor purging components, including one or more fans or pressurized fluid sources to militate against residue or vapors remaining in the device between samples or after use.

The subject device can be configured as a portable self-contained device that measures, in real time, the concentrations of hydroxyl-containing compounds, such as ethanol, methanol, and water in the hydrocarbon sample (e.g., gasoline) at an accuracy and precision comparable to the use of GC-MS by using two different types of sensor suites—a near-infrared (NIR) spectrometer and an electrochemical impedance (EI) spectrometer. These sensors can be combined with an advanced deep machine learning program that has attributes of artificial intelligence (AI). The use of a NIR spectrometer provides a means to identify functional groups in the sample for substances present in concentrations of at least 0.1% by volume; in this case, those substances having hydroxyl (—OH) groups as part of their molecular structure. The use of an EI spectrometer provides for a contrast between ethanol, methanol, water, and gasoline; for example, EI signal response is particularly sensitive to the relative permittivity of the substances in the fuel sample (e.g., 80.1 for water, 33.0 for methanol, 25.3 for ethanol, and ˜2.0 for gasoline dielectric constant (DC)).

The device is highly portable, where a user can easily carry or wheel about the device as desired, as the device can be configured with the noted components to provide self-contained package weighing about 15 lb. The device can also be configured to be resistant to drop, shock, and temperature changes, where embodiments include where the device can be ruggedized to provide at least an IP 56 rating, according to the ingress protection (IP) set by the International Electrotechnical Commission (IEC) to grade the resistance of electrical and electronic equipment enclosures against the intrusion of dust and liquids. Ergonomic aspects of the device can include handles and/or wheels positioned to allow for ease of carrying, transporting, and intuitive operation, thereby giving the user the flexibility to work wherever the samples are to be acquired.

The device can be equipped with a power source, such as a rechargeable lithium-ion battery, to provide several hours of operation (e.g., >8 hours of continuous use on a single charge). A standby mode can also be included to conserve battery life when the device is not in use. The device can house one or more status indicators (e.g., LEDs) that specify battery life, device state, and any potential errors. Additional features can include a built-in display touch screen that allows the user to view results in real-time, a data transfer port (e.g., USB port) for transferring data, on-board data storage that can store the results of one or many analyses, and wireless communication capability to communicate with local or remote networks (e.g., a 5G cellular network).

The device can be configured with a controller in communication with the various components, where the controller can include one or more custom printed circuit boards, and which can include a cloud-based storage application with an online portal. The controller can be configured to automatically sync to a specified database anytime it is connected to a wireless network or ethernet. Operation of the device, however, does not require a Wi-Fi, Bluetooth, or Ethernet connection to accurately perform chemometrics, as the sample results can be stored on-board the device and/or displayed in real-time. The online portal can provide access to review valuable analytics captured by the device and compare mass sets of data from various devices, allowing the user to analyze all sample data with unprecedented ease and flexibility.

A custom syringe pump can be provided in the device, where the pump can be configured to hold a variety of readily available medical grade syringes having various volumes; e.g., syringe bodies ranging from 10-25 mL. The syringe pump can utilize a luer-lock fitting to offer watertight, hassle-free syringe connection. The syringe pump can be connected to a wall-jet based flow cell using polyetheretherketone (PEEK) super flangeless nuts and PEEK ferrules for a high-pressure screw-in connection. The flow cell can include a micruX door for sample introduction, where the inlet flow is perpendicular to the working electrode surface. The flow cell assembly can incorporate standard fluidic ports (¼″-28 UNF) with an inlet channel of 0.8 mm I.D., providing low dead-volume operation (internal volume <5 μL) for high sensitivity electrochemical measurements with low sample requirements (microvolume <100 μL).

Screw-in connections can be used throughout the fluid-handling portions of the device assembly to carry the fluid sample from the temperature control block to the NIR optical flow cell and subsequently out of the assembly. The material type and geometric parameters of tubing materials employed can be tailored to suit the specific fluid sample being tested. For example, the tubing can have an ⅛″ OD and a 1/16″ ID allowing for a viscous fluid to be tested and moved therethrough without the need for a heavy-duty syringe pump.

The temperature-controlled flow cell can be configured to provide one or more preselected analysis parameters and differentiation between fluid samples. Physical properties or characteristics such as density and flash point have proportional relationships with temperature; therefore, the device can be configured to provide controlled sample temperatures to standardize data and increase reliability of the chemometric models. The fuel sample can be forced through a collection of flowthroughs at a uniform rate, where the sample will meet resistance temperature detectors (RTD). The data from the RTD can be fed to the controller, including a microcontroller unit (MCU), where the controller can initiate the thermoelectric devices based off the temperature region. The device can effectively standardize sample temperatures within an uncertainty range of +/−1° C.

The controller can be in communication with the various components of the device and can ensure the overall integrity and order of operations of the system. In certain embodiments, the controller can include an MCU programmed to manage a BME 680 chip, which measures environmental data such as ambient temperature, humidity, presence and concentration of volatile organics and ambient pressure. The MCU of the controller can also manage a lithographic test chip (LTC), which can provide precision voltage regulation for other components in the device, such as the temperature control flow cell, and collect temperature data using the inline sample temperature thermistors. Additionally, the MCU of the controller can control a 100 mm 1:150 linear actuator; this actuator can generate a pushing force of over 103 N, enabling the system to handle samples of varying viscosities. The controller can include a general processing unit (GPU) that provides processing capabilities to perform chemometrics in real time without the need to wait for post data processing. Operation of the MCU can be configured to use an advanced machine learning algorithm to continuously calibrate the device using backpropagation techniques, which can improve accuracy with each sample. In certain aspects, the controller can operate to maintain the state and security of the device while ensuring proper communication between all the various components and processing needs.

The NIR spectrometer can measure the absorption of light by the liquid sample analyzed within a preselected wavelength range, such as 1100 nm-2150 nm. This spectral region can provide characteristic information about the chemical composition of the sample; e.g., the presence and nature of —OH groups. This process is a non-destructive analytical technique that can determine the quality and composition of a wide range of materials, including gasoline, fuels, food, pharmaceuticals, and polymers. The NIR spectrometer can be deemed nontraditional as it is digital light-processing based, compact, and cost effective. The optical engine of the spectrometer can adopt a post-dispersive architecture, which can be composed of an entrance slit, a set of internal collimating lenses, a bandpass filter, a diffraction grating, a set of focusing lenses, a digital micromirror device (DMD), a set of light receiving lenses, and a sensor such as a single-element indium gallium arsenide (InGaAs) detector. Where an InGaAs detector is used, it can provide high sensitivity over the 900 nm-1700 nm wavelength range, which can be referred to as the shortwave infrared (SWIR) range. The digital light processing NIR spectrometer can provide a high level of spectral resolution comparable to a 512-pixel linear array detector at the same wavelength range, but at a fraction of the cost. Additionally, the digital light processing (DLP) based NIR spectrometer can offer enhanced flexibility in spectral selection, allowing for precise customization of the spectral range. Through preselected, customized, and/or real-time profiling, specific profiles can be developed that are tailored to efficiently scan the region(s) of interest for ethanol, methanol, water, and gasoline, leveraging the unique absorption peaks associated with each compound of interest. This can ensure use of the optimal spectral coverage required to make accurate predictions without unnecessary oversampling and power usage by the device.

The NIR spectrometer system can incorporate a high-performance broadband light source, such as a quartz-tungsten-halogen (QTH) lamp. This light source can be integrated into a housing equipped with an analog feedback control mechanism. The controller or the control system of the NIR spectrometer system can utilize a photodiode tuned to measure the spectral intensity emitted by the light source. Based on this feedback, the control system can adjust the supplied voltage to the light source, ensuring a consistent and stable spectral irradiance throughout the desired wavelength range; e.g., 360 nm to 2600 nm. The QTH lamp can be configured for low voltage and low power consumption without sacrificing spectral intensity or a steady state system. To complete the NIR spectrometer system, an optical flow path is selected to utilize small collimating and focusing lenses to maintain the signal from the light source across the flow cell and into the spectrometer.

The EI spectrometer measures the complex impedance of a sample, which relates to its electrical properties such as the permittivity, permeability, and electrical conductivity in the chemometric model. The EI spectrometer used with the controller (e.g., designed using Spectra CAD) can join a custom flow cell made from PEEK, designed to transport fluid directly onto the contact area of interdigitated ring array electrodes, without leaving remnant fluid behind. Furthermore, the flow cell can be accessibly housed within the device to make for ease of changing the electrodes. As the sample is processed, the EIS electrodes are awash in the flow of the sample allowing measurement of its electrical impedance and consequently the dielectric constant. The EI spectrometer can monitor the acidity, water content, and other properties of samples of fuels and lubricants. The EI spectrometer can be coupled with a frequency response analyzer that measures the impedance of the sample at different frequencies. The impedance of the sample can vary with its chemical composition, such that the resulting impedance spectrum can be analyzed in conjunction with Bode and Nyquist plots by a deep machine learning program to determine the chemical composition of the sample.

By combining these two techniques, NIR and EI spectroscopy, it is possible to use the present device to obtain a comprehensive understanding of the chemical and electrochemical behavior of the system, enabling deeper insights into the underlying mechanisms. This integrated approach provides several applications, such as in the optimization of electrochemical processes and the development of combined sensors for innovative analyses. The synergy between NIR spectroscopy and EIS provides a powerful tool in assaying samples in various applications, including material science, electrochemistry, and analytical chemistry, and thereby allows a user to gain a deeper understanding of such complex systems.

Components of the device can communicate and operate under direction of a controller configured using Spectra CAD. Various types and arrangements of controllers can be designed, programmed, and configured in various ways, including by the use of the Spectra CAD process available from HEKA (Melbourne, FL). Spectra CAD's process can include a deep machine learning program that can take input from a NIR spectrometer and an EI spectrometer in real time and can be configured to perform chemometrics to generate the outputs of physical property predictions, principal component composition, etc. Machine readable instructions, including non-transient machine-readable instructions, can be incorporated into the controller including one or more types of processors and memory modules. The controller and the instructions contained therein can be updated, stored, and can be dynamically modified in real time before, during, and/or after analysis of a sample. The controller can be configured with preset programs and modules for analyzing electrochemical systems, including those tailored for analyzing certain chemical species and applying certain chemometrics. The program can include certain components, including input data preprocessing, model training, and output prediction.

In certain embodiments, the device can operate as follows. Input data can be preprocessed to extract relevant information from raw NIR and EIS data. This can be performed in a 5-stage process including: data cleaning, normalization, transformation, splitting, and augmentation. The input data can be first checked for any errors, missing values, or outliers that may adversely affect the performance of the network. Any problematic data can either be removed or replaced with a reasonable substitute. Then the data can be normalized to ensure that each variable has a similar scale and distribution. Normalization can be done by scaling, centering, or standardizing the data; in certain embodiments, accurate models can be created using standardized data. Next, the data can be transformed by using the principal component analysis technique and, when faced with complex data, a Fourier transformation. The input data can then be divided into training, validation, and testing sets that can be used to cross reference the neural network. The validation set can be used to monitor the performance of the network during training, and the testing set can be used to evaluate the final performance of the network. Another step of the preprocessing can include data augmentation to increase the size and diversity of the data.

The machine learning model used in the program can include a deep neural network, which can include multiple layers of interconnected nodes that process the input data and output the desired predictions. The neural network can be trained using a large dataset of preprocessed NIR and EIS data collected using a controller configuration (e.g., designed by Spectra CAD) and developed using a standardized methodology, along with corresponding physical property measurements. During training, the neural network can learn the complex relationships between the input data and the physical properties of interest. Training a neural network can include iterative process that adjusts weights and biases of the network to minimize the difference between predicted outputs and actual outputs for a given set of input data. This process can be accomplished through a technique called backpropagation, which involves propagating the error signal backwards through the network to adjust weights and biases of each neuron.

Once the machine learning model is trained, the model can be used to make predictions on new input data. The input data can be preprocessed as before to ensure there is no bias, and the neural network can use its learned weights to generate predictions for the physical properties of interest at a predetermined threshold or accuracy. The controller configuration can apply the same ideology to allow accurate principal component composition analysis and conductivity measurement. These predictions can be used to gain deeper insights into the underlying electrochemical behavior of the device and overall system, enabling a user to optimize processes and tailor the electrochemical systems to provide improved performance. Operation and configuration of the controller in this manner can allow the controller (and the other device components) to be adapted to various applications in the petrochemical industry, including fuel quality control, process monitoring, and contamination detection. The device can be used to monitor the quality of fuels and lubricants in real-time, allowing for immediate corrective action to be taken, if necessary.

Examples of suitable extended wavelength near infrared (NIR) spectrometers include devices available from the InnoSpectra Corporation (Hsinchu, Taiwan). These devices can include the following modules: an extended reflective module, an extended fiber optic module, and an extended transmissive module. The extended reflective module can include the following specifications: model name NIR-M-R11; wavelength range 1350˜2150 nm; optical resolution typ. 12 nm @1530 nm LD; wavelength accuracy typ.±1 nm; signal-to noise ratio 5000:1 in 1 second scan; illumination module tungsten filament lamp with reflector, 0.7 W*4 slit size 0.025 mm; detector 1 mm extended InGaAs (uncooled); scan capability linear/Hadamard/slew scan communication interface: 1. USB and UART 2. BLE (optional); sensors ambient humidity and temperature sensors; power USB; dimensions 95.5×47.5×37.5 mm; weight <80 g. The extended fiber optic module can include the following specifications: model mame NIR-M-F11; wavelength range 1350˜2150 nm; optical resolution typ. 12 nm @1530 nm LD; detector 1 mm extended InGaAs (uncooled); scan capability linear/Hadamard/slew scan communication interface 1. USB and UART 2. BLE (Optional); sensors ambient humidity and temperature sensors; power USB; dimensions 59.5×47.5×24.5 mm; weight <60 g. The extended transmissive module can include the following specifications: model name NIR-M-T11; wavelength range 1350˜2150 nm; optical resolution typ. 12 nm @1530 nm LD wavelength accuracy typ.±1 nm; signal-to noise ratio 5000:1 in 1 second scan; illumination module tungsten filament lamp (0.7 W*1); cuvette holder path length=10 mm, Z-dimension=15 mm; slit size 0.025 mm; detector 1 mm extended InGaAs (uncooled); scan capability linear/Hadamard/slew scan; communication interface 1. USB and UART 2. BLE (optional); sensors ambient humidity and temperature sensors; power USB; dimensions 95.5×47.5×37.5 mm; weight <110 g.

Examples of an electro-impedance spectroscopy (EIS) module include devices provided by PalmSens (Houten, The Netherlands). These devices can include the EmStat Pico Module, which can operate as a dual channel potentiostat. The EmStat Pico is a stand-alone potentiostat module that can perform electrochemical measurements using one or more various types of electrochemical sensors. This dual-channel module can be integrated into the controller (e.g., printed circuit board (PCB)). The EIS module can include the following specifications: dimensions: 18×30×2.6 mm; dual channel (2×WE, 2×RE, 2×CE); EIS frequency range: 0.016 Hz to 200 kHz; full DC-potential range: −1.7 V to +2 V; current ranges: 100 nA to 5 mA.

To run a sample, the user can turn the device on, where the device can then prompt the user to collect a sample of 50 mL in a 60 mL syringe while the light source reaches a stable state. Next, the user can load the sample into the syringe dispenser and close the case. Once the light source comes to a steady-state, the device can prompt the user again to initiate testing. The system can be fully automated to run and analyze samples at the touch of a few buttons, where testing and results are available in only a few minutes. The device can also provide a time to stabilize the sample at a predetermined temperature.

The device can be configured with the following infrared spectroscopy and electrochemical impedance spectroscopy parameters. Infrared Spectroscopy: wavelength range 900-1700 nm (STD), 1350-2150 nm (EXT); wavelength accuracy 1 nm; signal-to-noise ratio: 5000:1 in a 1-second scan; allows easy differentiation between 1 percentile changes in test samples and can identify samples from a predetermined known database. Electrochemical Impedance Spectroscopy: Bode & Nyquist Plots from EIS are used along with absorbance results obtained in Infrared Spectroscopy to identify hydroxyl-containing compounds in the sample, which can be done in conjunction with a predetermined known database; the results can be used to identify the percentile of each component within a sample; current range 100 pA-100 mA; frequency range 0.016 Hz-200 kHz; DC-potential range −1.7 to +2 V; applied potential resolution 537 uV-932 uV; applied potential accuracy 0.5%; current accuracy 1%-2% depending on applied current range; current resolution 0.006% of selected current range; 2 channels (bi-potential mode); can allow for identification of hydroxyl-containing compounds in the sample.

### Examples

Example embodiments of the present technology are provided with reference to the several figures enclosed herewith.

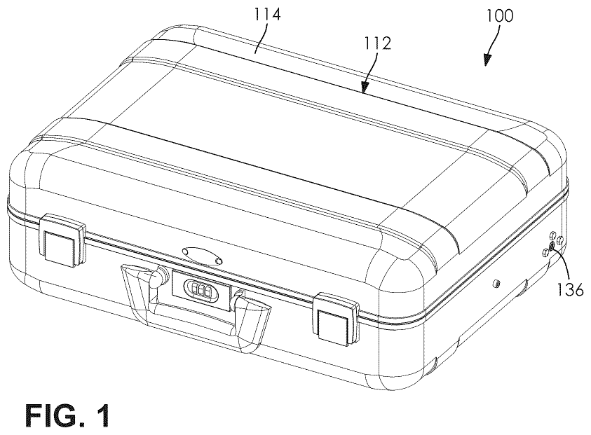

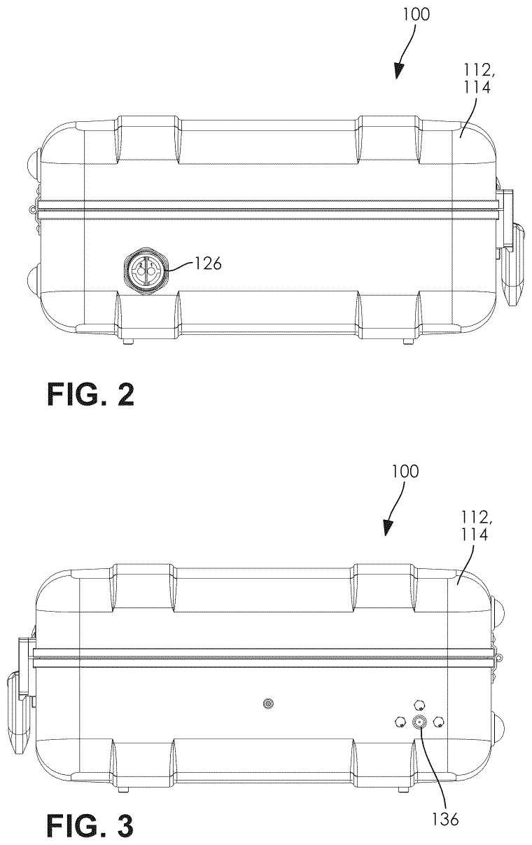

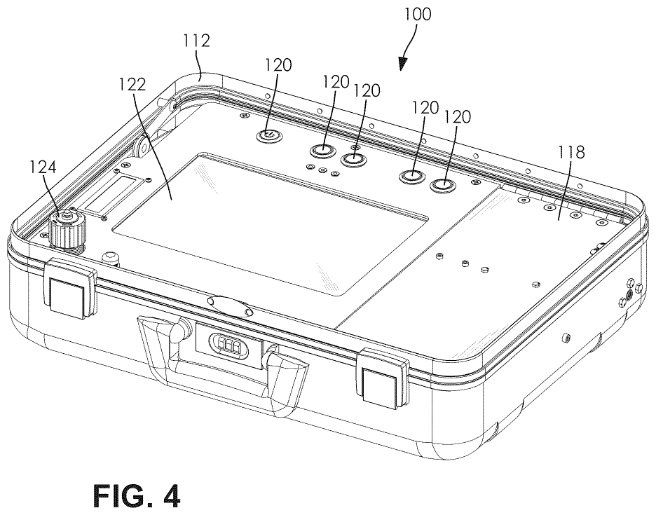

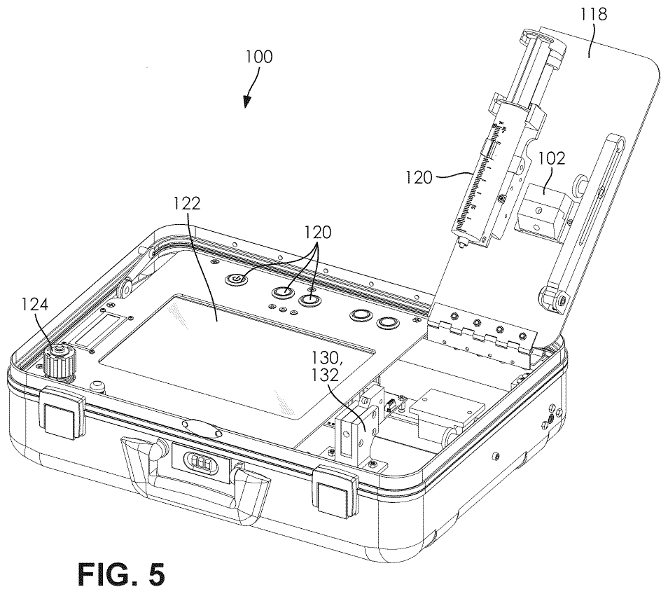

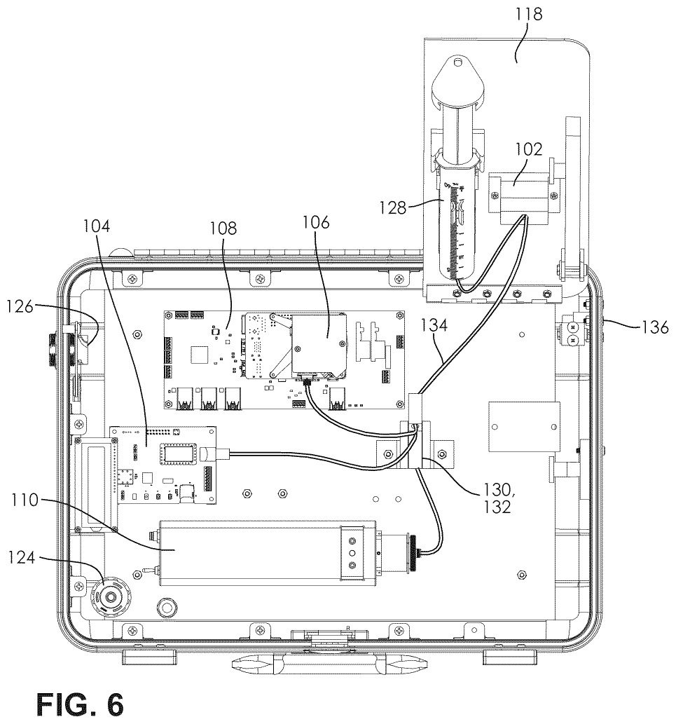

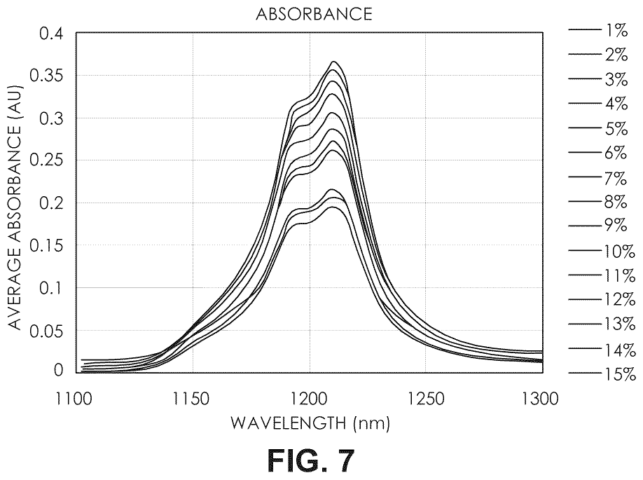

With reference to FIGS. 1-6, an embodiment of a device 100 for measuring hydroxyl-containing compounds in a hydrocarbon sample is shown. The device 100 includes a sample inlet 102 that receives the hydrocarbon sample for analysis. A near-infrared (NIR) spectrometer 104 is positioned to receive the sample and is configured to provide a first signal relating to identification of hydroxyl-containing compounds in the hydrocarbon sample. An electrochemical impedance (EI) spectrometer 106 is arranged to receive the sample and provide a second signal relating to differentiation of hydroxyl-containing compounds in the hydrocarbon sample. A controller 108 is communicatively coupled to both spectrometers and configured to receive the first signal from the NIR spectrometer 104 and the second signal from the EI spectrometer 106, where the controller 108 processes these signals to determine both the presence and amount of hydroxyl-containing compounds in the hydrocarbon sample. A quartz-tungsten-halogen (QTH) light source 110 with analog feedback control can be provided to generate stable spectral irradiance for the NIR measurements.

The above components are located within a housing 112, which is depicted herein in a configuration similar to a briefcase. The housing 112 includes a lid 114 that can be opened to access the device 100 components. Upon opening the lid 114, an interior panel 116 is revealed containing a hinged access panel 118. The interior panel 118 includes indicator LEDs 120 for displaying device status and battery life, and a built-in display touchscreen interface 122 for real-time results viewing. An ethernet port 124 and power receptacle 126 can be provided for network connectivity and power supply, including recharging of an onboard battery. The hinged interior panel 116 can be lifted to expose a syringe pump 128 configured to hold medical grade syringes (10-25 mL), which is fluidly coupled to the inlet 102.

A temperature-controlled flow cell 130 can be configured to transport fluid from the inlet 102 to the NIR spectrometer 104, the EI spectrometer 106, and light source 110. The flow cell 130 can include a linear actuator 132 capable of generating over 103 N of pushing force configured to transfer the hydrocarbon sample from the inlet 102 to the NIR and EI spectrometers 104, 106. The device 100 can incorporate polyetheretherketone (PEEK) tubing 134 and connections throughout the fluid-handling portions. The tubing 134 can connect the inlet 102 to the NIR spectrometer 104 and EI spectrometer 106. A drain 136 can be provided in the housing for sample removal.

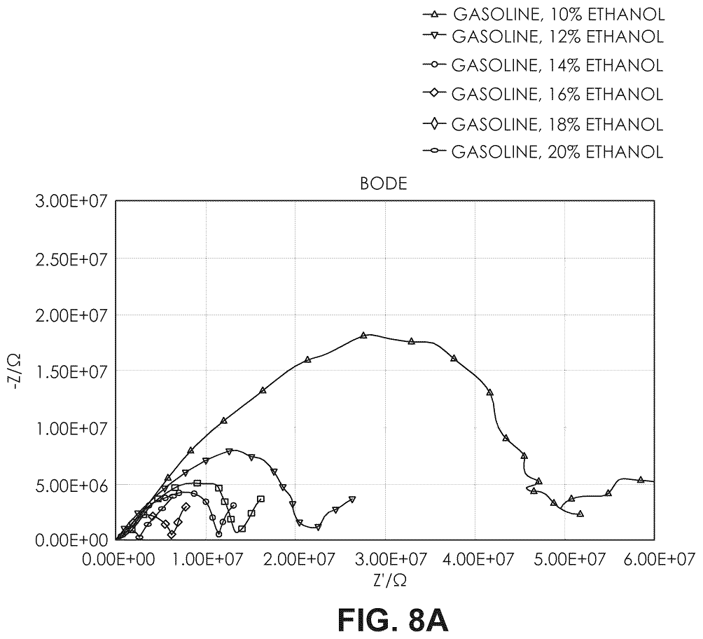

FIG. 7 graphically depicts example average absorbance (AU) values measured by the NIR spectrometer 104 for a hydrocarbon sample analyzed across an infrared light wavelength range of 1100-1300 nanometers. The absorbance measurements in this wavelength range enable identification of hydroxyl-containing compounds present in the sample, as NIR spectroscopy provides a means to identify functional groups for substances present in concentrations of at least 0.1% by volume, particularly those substances having hydroxyl (—OH) groups as part of their molecular structure. When analyzed in conjunction with the EI spectrometer 106 data, these absorbance measurements allow the controller 108 to determine both the presence and amounts of hydroxyl-containing compounds like ethanol, methanol, and water in the hydrocarbon sample with high precision.

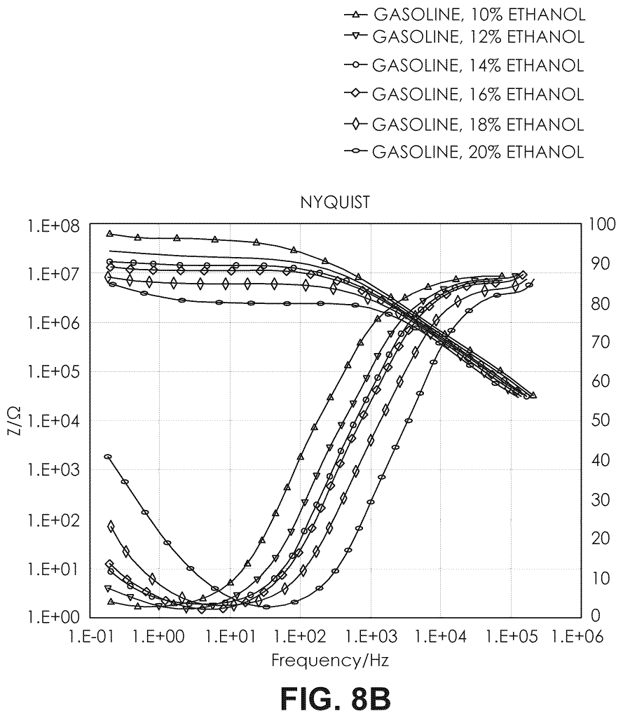

With reference to FIGS. 8A and 8B, graphical representations of Bode and Nyquist plots are shown for gasoline samples containing varying ethanol concentrations ranging from 10% to 20%. These plots demonstrate the electrochemical impedance spectroscopy (EIS) data collected by the device 100, where the EIS signal response is particularly sensitive to the relative permittivity of the substances in the fuel sample. The Bode and Nyquist plots, when analyzed in conjunction with the absorbance results obtained from NIR spectroscopy, enable the controller 108 to identify and quantify the hydroxyl-containing compounds present in the sample. This analytical approach leverages the significant differences in dielectric constants between the compounds of interest (80.1 for water, 33.0 for methanol, 25.3 for ethanol, and approximately 2.0 for gasoline), allowing for precise differentiation and measurement of these components within the fuel sample.

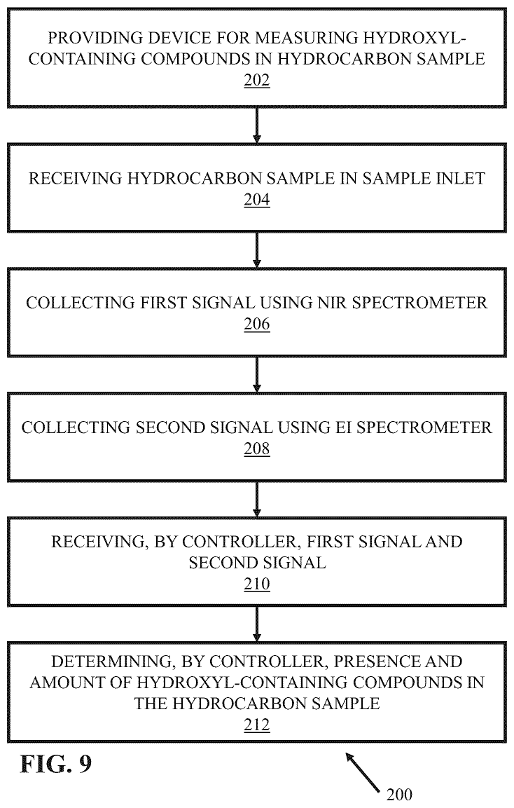

FIG. 9 graphically depicts a method 200 for measuring hydroxyl-containing compounds in a hydrocarbon sample. A step 202 of providing includes furnishing a device with a sample inlet, a near-infrared (NIR) spectrometer configured to provide a first signal relating to identification of hydroxyl-containing compounds, an electrochemical impedance (ELI) spectrometer configured to provide a second signal relating to differentiation of hydroxyl-containing compounds, and a controller configured to receive and process the signals. A step 204 of receiving includes introducing the hydrocarbon sample through the inlet 102 which directs the sample to a temperature-controlled flow cell 130. A step 206 of collecting the first signal utilizes the NIR spectrometer to identify functional groups for substances present in concentrations of at least 0.1% by volume, particularly focusing on compounds containing hydroxyl (—OH) groups. A step 208 of collecting the second signal employs the EI spectrometer, leveraging its sensitivity to the relative permittivity of substances in the fuel sample, with particular differentiation between compounds like water (80.1), methanol (33.0), ethanol (25.3), and gasoline (˜2.0). A step 210 of receiving involves the controller acquiring both the first signal from the NIR spectrometer and the second signal from the EI spectrometer. Finally, a step 212 of determining involves the controller utilizing machine learning algorithms to process the collected signals, enabling determination of both the presence and quantities of hydroxyl-containing compounds in the hydrocarbon sample to within about 0.1% by mass.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A device for measuring a hydroxyl-containing compound in a hydrocarbon sample, comprising:
a sample inlet for receiving the hydrocarbon sample;
a near-infrared (NIR) spectrometer configured to provide a first signal relating to identification of hydroxyl-containing compounds in the hydrocarbon sample;
an electrochemical impedance (EI) spectrometer configured to provide a second signal relating to differentiation of hydroxyl-containing compounds in the hydrocarbon sample; and
a controller configured to receive the first signal from the NIR spectrometer and the second signal from the EI spectrometer, the controller configured to determine a presence and an amount of the hydroxyl-containing compound in the hydrocarbon sample.

2. The device of claim 1, further comprising a temperature control means configured to control a temperature of the hydrocarbon sample.

3. The device of claim 1, further comprising a syringe configured to be fluidly coupled to the sample inlet.

4. The device of claim 1, further comprising:
a power source;
a light source;
an actuator mechanically coupled to the sample inlet and configured to transfer the hydrocarbon sample from the sample inlet to the NIR spectrometer and the EI spectrometer;
an electronic user interface;
a data port configured to provide input/output signals with the controller; and
a housing.

5. The device of claim 1, wherein the hydroxyl-containing compound in the hydrocarbon sample includes a member selected from a group consisting of methanol, ethanol, water, and combinations thereof.

6. The device of claim 1, wherein the NIR spectrometer is configured to measure absorption of light by the hydrocarbon sample within a wavelength range of 1100 nm to 2150 nm.

7. The device of claim 1, further comprising a temperature control means configured to standardize sample temperatures within an uncertainty range of ±1° C.

8. The device of claim 1, further comprising a quartz-tungsten-halogen lamp configured to provide stable spectral irradiance.

9. The device of claim 1, further comprising a rechargeable lithium-ion battery.

10. The device of claim 1, further comprising a built-in display touch screen configured to view results in real-time.

11. The device of claim 1, wherein the device is configured to determine concentrations of hydroxyl-containing compounds to within about 0.1% by mass.

12. The device of claim 1, wherein the device is configured to be resistant to drop, shock, and temperature changes with an IP 56 rating.

13. The device of claim 1, further comprising:
a temperature control means configured to standardize sample temperatures within an uncertainty range of ±1° C.;
a syringe configured to be fluidly coupled to the sample inlet and configured to hold between 10-25 mL;
a rechargeable lithium-ion power source configured to provide over 8 hours of continuous operation;
a quartz-tungsten-halogen light source configured to provide stable spectral irradiance;
an actuator configured to generate over 103 N of pushing force to transfer the hydrocarbon sample from the sample inlet to the NIR spectrometer and the EI spectrometer
a built-in display touch screen interface configured to view results in real-time;
a data port configured to provide input/output signals with the controller;
a housing configured with an IP 56 rating for protection against dust and water intrusion;
wherein the NIR spectrometer is configured to measure absorption of light by the hydrocarbon sample within a wavelength range of 1100 nm to 2150 nm;
wherein the device is configured to determine concentrations of the hydroxyl-containing compound to within about 0.1% by mass;
wherein the hydroxyl-containing compound in the hydrocarbon sample includes a member selected from a group consisting of methanol, ethanol, water, and combinations thereof.

14. The device of claim 1, further comprising status indicator LEDs configured to specify battery life and device state.

15. A method for measuring a hydroxyl-containing compound in a hydrocarbon sample, comprising:
providing a device for measuring hydroxyl-containing compounds in the hydrocarbon sample according to claim 1;
receiving the hydrocarbon sample in the sample inlet;
collecting the first signal using the NIR spectrometer;
collecting the second signal using the EI spectrometer;
receiving, by the controller, the first signal and the second signal; and
determining, by the controller, the presence and the amount of the hydroxyl-containing compound in the hydrocarbon sample.

16. The method of claim 15, wherein the hydroxyl-containing compound in the hydrocarbon sample includes a member selected from a group consisting of methanol, ethanol, water, and combinations thereof.

17. The method of claim 15, wherein the device further comprises a temperature control means configured to control a temperature of the hydrocarbon sample, and the method further comprises a step of controlling the temperature of the hydrocarbon sample.

18. The method of claim 15, wherein the device further comprises a syringe configured to be fluidly coupled to the sample inlet, and the method further comprises a step of dispensing, via the syringe, the hydrocarbon sample through the inlet.

19. The method of claim 15, wherein the device further comprises:
a power source;
a light source, and the method further comprises a step of measuring, by the light source, the absorption of light by the sample analyzed within a preselected wavelength range;
an actuator mechanically coupled to the sample inlet and configured to transfer the hydrocarbon sample from the sample inlet to the NIR spectrometer and the EI spectrometer, and the method further comprises a step of dispensing, via the actuator, the hydrocarbon sample;
an electronic user interface;
a data port configured to provide input/output signals with the controller; and
a housing.

20. The method of claim 15, wherein the device further comprises:
a temperature control means configured to standardize sample temperatures within an uncertainty range of ±1° C., and the method further comprises a step of controlling the temperature of the hydrocarbon sample;
a syringe configured to be fluidly coupled to the sample inlet and configured to hold between 10-25 mL, and the method further comprises a step of dispensing, via the syringe, the hydrocarbon sample through the inlet;
a rechargeable lithium-ion power source configured to provide over 8 hours of continuous operation;
a quartz-tungsten-halogen light source configured to provide stable spectral irradiance;
an actuator configured to generate over 103 N of pushing force to transfer the hydrocarbon sample from the sample inlet to the NIR spectrometer and the EI spectrometer
a built-in display touch screen interface configured to view results in real-time;
a data port configured to provide input/output signals with the controller;
a housing configured with an IP 56 rating for protection against dust and water intrusion;
wherein the NIR spectrometer is configured to measure absorption of light by the hydrocarbon sample within a wavelength range of 1100 nm to 2150 nm;
wherein the device is configured to determine concentrations of hydroxyl-containing compounds to within about 0.1% by mass;
wherein the hydroxyl-containing compounds in the hydrocarbon sample include a member selected from a group consisting of methanol, ethanol, water, and combinations thereof.
